(19) United States
(12) Patent Application Publication
(10) Pub. No.: US 2025/0303181 A1
(43) Pub. Date: Oct. 2, 2025

(54) LEAD ADAPTER FOR IMPLANTABLE PULSE GENERATOR AND METHOD OF REPLACING AN IMPLANTABLE PULSE GENERATOR

(71) Applicant: The Alfred E. Mann Foundation for Scientific Research, Valencia, CA (US)

(72) Inventors: Joseph L. Calderon, Santa Clarita, CA (US); Brian Michael Shelton, Ventura, CA (US); Neil Talbot, La Crescenta, CA (US)

(21) Appl. No.: 19/094,381

(22) Filed: Mar. 28, 2025

Related U.S. Application Data
(60) Provisional application No. 63/572,083, filed on Mar. 29, 2024.
(60) Provisional application No. 63/572,091, filed on Mar. 29, 2024.

(51) Int. Cl. A61N 1/375; A61N 1/372; A61N 1/378; G16H 40/63

(52) U.S. Cl. CPC A61N 1/3752; A61N 1/37247; A61N 1/378; G16H 40/63

(57) ABSTRACT

A lead splitter adapter for an implantable pulse generator includes a plug portion configured to plug into a receptacle of an implantable pulse generator. The plug portion includes electrical contacts, and at least two of the electrical contacts, a first electrical contact from within the first lead receptacle and a second electrical contact from the second lead receptacle, which first and second electrical contacts are shunted by an electrical connection that includes at least one of a resistor, capacitor or inductor.

### CROSS-REFERENCE TO RELATED APPLICATION(S)

The present application claims priority to and the benefit of U.S. Provisional Application No. 63/572,083, filed Mar. 29, 2024, and U.S. Provisional Application No. 63/572,091, filed Mar. 29, 2024, the entire content of both of which is incorporated herein by reference.

### BACKGROUND

### 1. Field

The present disclosure relates to embodiments of lead adapters for implantable pulse generators.

### 2. Description of the Related Art

Implantable pulse generators (IPGs) are utilized for a variety of therapeutic purposes, such as treating heart conditions, swallowing disorders onset by a stroke, sleep apnea, chronic back pain, and other medical conditions. IPGs are connected to one or more electrical stimulation leads to deliver electrical stimulation to a portion of the patient depending on the condition being treated.

The above information disclosed in this Background section is only to enhance understanding of background information pertaining to the present disclosure and may contain information that does not constitute prior art.

### SUMMARY

The present disclosure relates to various embodiments of a lead splitter adapter. In one embodiment, the lead splitter adapter includes a plug portion configured to plug into a lead receptacle of an implantable pulse generator. The plug portion includes electrical contacts. The lead splitter adapter also includes a first receptacle portion and a second receptacle portion connected to the plug portion. Each of the first receptacle portion and the second receptacle portion includes electrical contacts. The lead splitter adapter also includes at least one short between one of the electrical contacts of the first receptacle portion and one of the electrical contacts of the second receptacle portion.

The present disclosure also relates to various embodiments of a stimulator system. In one embodiment, the stimulator system includes an implantable pulse generator including a header, a lead receptacle in the header, electrical contacts in the lead receptacle, a processor, a non-volatile memory device, and a power supply. The system also includes a lead splitter adapter configured to connect at least one electrical stimulation lead to the implantable pulse generator. The lead splitter adapter includes a proximal plug portion configured to extend into the lead receptacle in the header of the implantable pulse generator. The plug portion includes electrical contacts. The lead splitter adapter also includes first and second receptacles, each of which are configured to accept the proximal connector end of a stimulation lead. The first and second receptacles include electrical contacts. The non-volatile memory device includes instructions which, when executed by the processor, cause the implantable pulse generator to determine at least one of an impedance, a resistance, a capacitance, or an inductance between two of the electrical contacts of the plug portion and to determine a configuration of the at least one electrical stimulation lead based on the impedance, the resistance, the capacitance, or the inductance.

In one embodiment, the lead splitter adapter has six electrical contacts at the proximal plug portion and has first and second receptacles, each having six electrical contacts within each receptacle. Conventionally, an IPG with six-electrical contacts is connected directly to a stimulation lead with six electrical contacts at the proximal, connector end and six electrode contacts on the distal end. In one embodiment, the lead splitter design disclosed herein allows two six-electrode contact stimulation leads to be connected to the IPG having six electrical contacts within a lead receptacle. This is accomplished by having four electrical contacts on the splitter proximal plug or connector portion be electrically connected to four individual electrical contacts in the receptacles: two in the first splitter lead receptacle and two in the second splitter lead receptacle. An electrical connection can be made between one of the electrical contacts in the first splitter lead receptacle and one of the electrical contacts in the second splitter lead receptacle. This electrical connection includes at least one of a resistor, capacitor, and an inductor. The impedance of the electrical connection can be detected or measured. The presence of this electrical connection and detected signature impedance (or alternatively, resistance, inductance, or capacitance) can be used to distinguish between when the lead splitter is connected to an IPG as opposed to the IPG being connected directly to a stimulation lead. The remaining two electrical contacts at the splitter plug or connector portion electrically connect to two or more of the electrical contacts in the splitter receptacles. Some electrical contacts between the first and second receptacles are shorted. A method is disclosed for detecting the electrical connection in order to distinguish when the IPG is connected to the lead splitter or when the IPG is connected directly to a stimulation lead. In another embodiment, the lead splitter design disclosed also allows two six-electrode contact stimulation leads to be connected to the IPG having six electrical contacts within a lead receptacle. This is accomplished by using a lead splitter that has six electrical contacts at the proximal plug portion. This splitter has two lead receptacles, a first splitter lead receptacle and a second splitter lead receptacle, in the distal receptacle portion of the lead splitter. The first splitter lead receptacle has at least three electrical contacts and the second splitter lead receptacle has at least three electrical contacts. Each of the six electrical contacts at the splitter plug portion is connected to one of the electrical contacts in the first splitter lead receptacle or to one of the electrical contacts in the second splitter lead receptacle. An electrical connection can be made between one electrical contact in the first splitter lead receptacle and one electrical contact in the second first splitter lead receptacle. This electrical connection includes at least one of a resistor, capacitor, or an inductor. The impedance or resistance of the electrical connection can be detected or measured. The presence of this electrical connection and detected signature impedance (or alternatively, resistance, inductance or capacitance) can be used to distinguish when the lead splitter is connected to an IPG, as opposed to the IPG being connected directly to a stimulation lead. A method is disclosed for detecting the electrical connection in order to distinguish between when the IPG is connected to the lead splitter or when the IPG is connected directly to a stimulation lead. In addition, a method is disclosed for automatically configuring the IPG stimulation settings and wirelessly connected clinician programmers or patient programmers so that they automatically display the correct lead configurations in the graphical user interfaces (GUI) to set which IPG electrical contacts positive or negative of OFF once the lead configuration is detected as either: (1) the IPG connected to a lead splitter, which in turn is connected to two stimulation leads or (2) the IPG is connected directly to a stimulation lead.

This summary is provided to introduce a selection of features and concepts of embodiments of the present disclosure that are further described below in the detailed description. This summary is not intended to identify key or essential features of the claimed subject matter, nor is it intended to be used in limiting the scope of the claimed subject matter. One or more of the described features or tasks may be combined with one or more other described features or tasks to provide a workable device or method.

### BRIEF DESCRIPTION OF THE DRAWINGS

The features and advantages of embodiments of the present disclosure will become more apparent by reference to the following detailed description when considered in conjunction with the following drawings. In the drawings, like reference numerals are used throughout the figures to reference like features and components. The figures are not necessarily drawn to scale.

FIG. 1 is a schematic view of a stimulation system according to one embodiment of the present disclosure including an implantable pulse generator and an electrical stimulation lead;

FIG. 2 is a block diagram of the embodiment of the implantable pulse generator illustrated in FIG. 1;

FIG. 3 is a schematic view of a stimulation system according to another embodiment of the present disclosure including an implantable pulse generator, a pair of electrical stimulation leads, and a lead splitter connecting the electrical stimulation leads to the implantable pulse generator;

FIG. 4 is a block diagram of the embodiment of the implantable pulse generator illustrated in FIG. 3;

FIG. 5A is a schematic diagram of the lead splitter according to the embodiment illustrated in FIG. 3;

FIG. 5B is a schematic diagram of yet another embodiment of the lead splitter according to the embodiment illustrated in FIGS. 3; and

FIG. 6 is a flowchart illustrating tasks of a method for determining between lead configurations (1) the IPG is connected to a single stimulation lead or (2) the IPG is connected to a lead splitter adapter, which in turn, is connected to two stimulation leads.

### DETAILED DESCRIPTION

The present disclosure relates to various embodiments of a stimulation system including an implantable pulse generator (IPG), at least one stimulation lead having at least one electrode, and a stimulation system that employs a lead splitter adapter (also referred to as an “adapter”, a “splitter” or “lead splitter”) which permits the IPG to be used with two stimulation leads.

The IPG can be used in a number of different system lead configurations. A conventional system configuration includes an IPG used with a stimulation lead that is designed to be used together. For example, the IPG may be a six-channel stimulation system with a single lead receptacle or port. As used herein, the term “six-channel” means that the lead receptacle or port in the IPG has six electrical contacts, where each electrical contact may function as a negative or positive contact or can be turned off (OFF) so that it is not functioning as either a negative or positive contact. The receptacle can be connected to a stimulation lead having six electrical contacts at the proximal, plug or connector end of the lead and six electrode contacts at the distal end of the lead. The distal end of the lead may be, for example, a linear electrode having an array of six electrode contacts or a nerve cuff electrode having six electrode contacts. When the IPG and stimulation lead are designed to be dimensionally compatible and has the same number of electrical contacts in the IPG lead receptacle and the electrical contacts on the connector or plug end of the stimulation lead, no lead adapter is required.

In one or more embodiments, two or more electrical contacts located in the two receptacles of the lead splitter adapter are shorted (shunted). The IPG is configured to measure or determine the impedance through the electrical contacts at the plug end of the splitter and to utilize this information to detect or determine the system configuration of: (1) an IPG connected to a single stimulation lead or (2) an IPG connected to a splitter and two stimulation leads. In one or more embodiments, the splitter may be shorted (shunted) between two or more electrical contacts in the two receptacles of the lead splitter, and the IPG may be configured to measure or determine the resistance, inductance, and/or capacitance between the electrical contacts on the plug portion of the lead splitter or the plug portion of the stimulation lead and to utilize this information to detect or determine the configuration of the lead(s) and/or the lead splitter. In this manner, the resistance, capacitance, or inductance of the electrical contacts at the proximal plug portion of the splitter encodes an identification (ID) used by the IPG to indicate the configuration of the lead splitter and/or the lead(s).

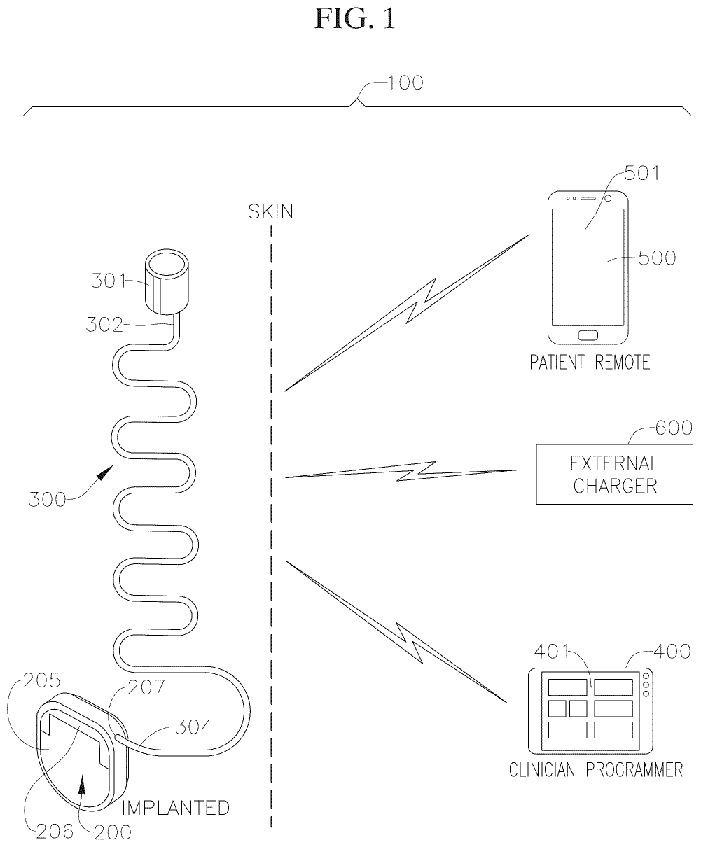

FIG. 1 depicts an example of a conventional stimulation system using a single stimulation lead. A stimulation system 100 is configured to treat a patient via electrical neurostimulation, e.g., hypoglossal nerve stimulation, or spinal cord stimulation according to one embodiment of the present disclosure. System 100 includes an implantable pulse generator (IPG) 200, an electrical stimulation lead 300, a clinician programmer (CP) device 400 and a patient remote (PR) device 500 each electronically coupled to (i.e., in wireless RF communication with) the IPG 200. The CP device 400 and the PR device 500 are each configured to bi-directionally communicate with the IPG 200 through the patient's skin (i.e., transcutaneously). The CP device 400 is configured to set one or more operating parameters or settings of the IPG 200. In one or more embodiments, the stimulation system 100 may also include an external charger 600 configured to wirelessly (e.g., inductively) charge the IPG 200 through the patient's skin.

The electrical stimulation lead 300 includes a known electrode 301 (e.g., a cuff electrode, as shown in FIG. 1, or a helical cuff electrode, a linear electrode, a percutaneous electrode, or a spinal cord paddle electrode, each electrode having two or more electrode contacts) at a distal end 302 of the electrical stimulation lead 300 to periodically deliver an electric current pulse for a variety of therapeutic treatments for the patient, such as neurostimulation, and/or spinal cord stimulation. The type or kind of the electrodes 301 may be selected based on the location and the type nerves stimulated (e.g., a cuff electrode to stimulate a nerve bundle, such as the hypoglossal nerve or the vagus nerve; a linear lead to stimulate the brain, or a spinal cord lead, such as a paddle or a linear lead, to stimulate the spinal cord). The electrical stimulation lead 300 also includes a plurality of electrical contacts 303 at a proximal end 304 of the electrical stimulation lead 300. The electrical contacts 303 may include any suitably conductive metal, such as titanium or a stainless-steel alloy. The IPG 200 and the electrical stimulation lead 300 may be implanted in any suitable locations in the patient depending on the therapeutic treatment delivered by the system 100. In one or more embodiments, the IPG 200 may be implanted in a subcutaneous pocket in the upper chest of the patient, and the electrical stimulation lead 300 may extend from the IPG 200 via the neck to at least one of the patient's nerves, such as the vagus nerve, the hypoglossal nerve, or into the abdomen at the phrenic nerve which innervates the diaphragm.

The CP device 400 and the PR device 500 each include a display 401, 501 (e.g., a light-emitting diode (LED) display), respectively configured to display a graphical user interface (GUI). The GUI may display various information related to the stimulation mode (or stimulation parameters) of the IPG 200 and/or the configuration of the electrical stimulation lead 300, among other information.

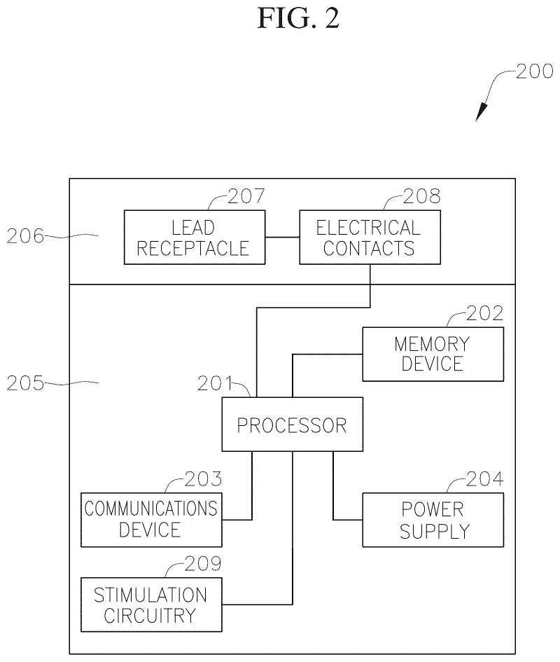

FIG. 2 shows a block diagram of an embodiment of the IPG 200. The IPG 200 includes a processor (e.g., a processing circuit) 201, a non-volatile memory device 202 (e.g., flash memory), a communications device 203 (e.g., a receiver and a transmitter, or a transceiver), and a power supply 204 (e.g., a primary battery or an inductively chargeable rechargeable battery). The communications device 203 provides wireless communication links through the skin of the patient to the CP device 400 and the PR device 500. Wireless links may include Bluetooth™, Bluetooth Low Energy or other protocols with suitable authentication and encryption to protect patient data. In one or more embodiments, the non-volatile memory device 202, the communications device 203, and the power supply 204 are in communication with each other over the processor 201. Additionally, in the illustrated embodiment, the processor 201, the non-volatile memory device 202, the communications device 203, and the power supply 204 are housed in a housing or a case 205. The case 205 includes a header 206 (e.g., a top epoxy part) and a lead receptacle 207 (e.g., a port or opening) in the header 206. The header 206 also includes a plurality of electrical contacts 208 (e.g., annular (ring-shaped) contacts) in the lead receptacle 207. The electrical contacts 208 may include any suitably conductive metal, such as titanium or a stainless-steel alloy. Typically, the electrical contacts inside the lead receptacle 207 in the IPG header 206 are canted coil springs that are shaped into rings which accept the plug or connector end of the lead. An example of such a canted coil spring connector is described in U.S. Pat. No. 10,535,945, the entire content of which is incorporated herein by reference. In one embodiment, the header 206 of the IPG 200 includes six electrical contacts 208 in the lead receptacle 207, although in one or more embodiments the header 206 of the IPG 200 may include any other suitable number of electrical contacts 208 in the lead receptacle 207. The electrical contacts 208 are connected to dedicated stimulation circuitry 209 in the IPG 200 that provides stimulation pulses as controlled by the processor 201.

In one or more embodiments, the lead receptacle 207 may accommodate (e.g., receive) the proximal end 304 of the electrical stimulation lead 300. For instance, the lead receptacle 207 may accommodate (e.g., receive) the proximal end 304 of the electrical stimulation lead 300 in an embodiment in which the number of electrical contacts at the proximal end 304 of the electrical stimulation lead 300 is equal to the number of electrical contacts 208 in the lead receptacle 207 of the IPG 200.

For clarity, as used in this disclosure, an IPG 200 with a six-channel stimulation system means that the lead receptacle 207 has six electrical contacts 208 which are independently programmable and can deliver a stimulus pulse through each contact 208. In a conventional stimulation system, the IPG 200 is connected to a stimulation lead 300 having six electrode contacts. In a bipolar stimulation mode, at least one of the electrode contacts on the stimulation lead 300 is selected as a cathode and at least one electrode contact is selected as a return anode. Or in some embodiments, when the IPG metal portion of the housing 205 functions as a return, indifferent, anode, any one or more of the electrode contacts on the stimulation lead 300 may function as a cathode. This latter mode of stimulation, where the IPG housing 205 functions as the return anode, is known as “monopolar” or “unipolar” stimulation.

When a lead splitter is used and the IPG is programmed in a unipolar stimulation mode with the IPG housing functioning as a return anode, any one or all of the electrode contacts in the first or second stimulation leads which are electrically connected to the IPG can be selected as a cathode. Any unused electrode contacts in the first and second stimulation leads must be programmed to be “OFF” or in inactive mode.

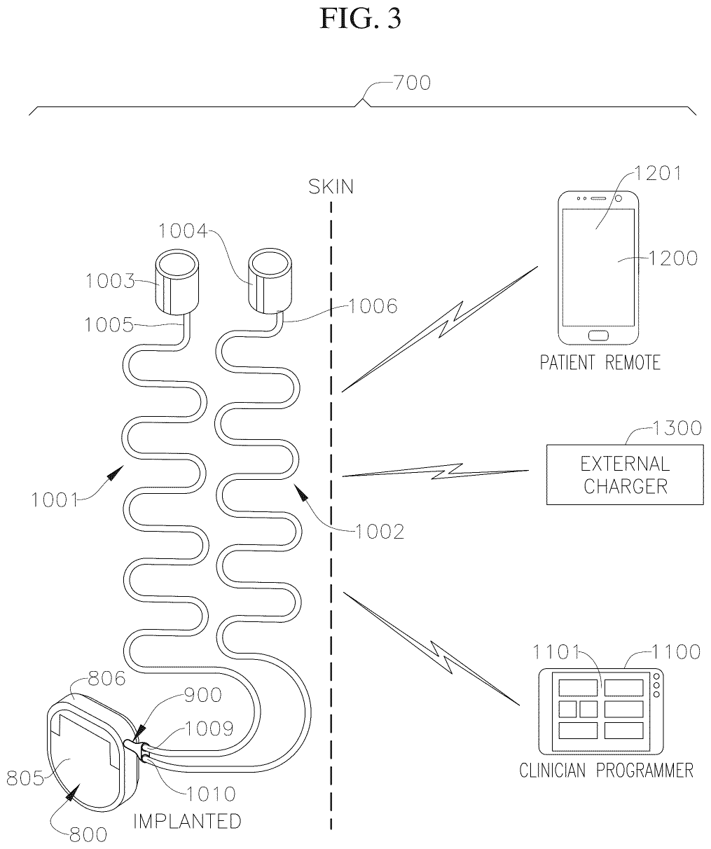

FIG. 3 shows a stimulation system 700 with an implantable pulse generator (IPG) 800 having only a single lead receptacle, a lead splitter adapter 900, and a pair of electrical stimulation leads 1001, 1002 (i.e., a first electrical stimulation lead 1001 and a second electrical stimulation lead 1002). The stimulation system 700 is configured to treat a patient via electrical stimulation, such as neurostimulation at two different nerve sites, e.g. vagus nerve stimulation, hypoglossal nerve stimulation, either bilaterally or a combination of different nerves such as vagus and hypoglossal nerve. The lead splitter adapter 900 is configured to connect the first and second electrical stimulation leads 1001, 1002 to the IPG 800. In one or more embodiments, the system 700 also includes a clinician programmer (CP) device 1100 and a patient remote (PR) device 1200 each electronically coupled to (i.e., in wireless RF communication with) the IPG 800. The CP device 1100 and the PR device 1200 are each configured to bi-directionally communicate with the IPG 800 through the patient's skin (i.e., transcutaneously). The CP device 1100 and the PR device 1200 each include a display 1101, 1201 (e.g., a light-emitting diode (LED) display), respectively configured to display a graphical user interface (GUI). The GUI may display various information related to the stimulation mode (or stimulation parameters) of the IPG 800 and/or the configuration of the first and second electrical stimulation leads 1001, 1002, among other information. In one or more embodiments, the stimulation system 700 may also include an external charger 1300 configured to wirelessly (e.g., inductively) charge the IPG 700 through the patient's skin.

The first and second electrical stimulation leads 1001, 1002 may include various types of electrodes 1003, 1004, respectively (e.g., cuff electrodes, linear electrodes, or a spinal cord paddle electrodes) at a distal end 1005, 1006, respectively, of the electrical stimulation lead 1001, 1002, respectively, to periodically deliver an electric current pulse for a variety of therapeutic treatments for a patient with neurostimulation. The type or kind of the electrodes 1003, 1004 may be selected based on the location and the type nerves stimulated (e.g., a cuff electrode to stimulate a nerve bundle, such as the hypoglossal nerve, phrenic, or the vagus nerve; or a spinal cord lead, such as a paddle or a linear lead, to stimulate the spinal cord). The first and second electrical stimulation leads 1001, 1002 also include a plurality of electrical contacts 1007, 1008, respectively, at a proximal end 1009, 1010, respectively, of the first and second electrical stimulation leads 1001, 1002. The electrical contacts 1007, 1008 may include any suitably conductive metal, such as titanium or a stainless-steel alloy.

The IPG 800 and the first and second electrical stimulation leads 1001, 1002 may be implanted in any suitable locations in the patient depending on the therapeutic treatment delivered by the system 700. In one or more embodiments, the IPG 800 may be implanted in a subcutaneous pocket in the upper chest of the patient. In one or more embodiments, the first and second electrical stimulation leads 1001, 1002 may be configured to provide unilateral stimulation to two different locations in the patient. For example, in one or more embodiments, the first electrical stimulation lead 1001 may extend from the IPG 800 to the right branch of the patient's hypoglossal nerve (e.g., a proximal or distal portion of the right branch of the hypoglossal nerve) and the second electrical stimulation lead 1002 may extend from the IPG 800 to the right side of the patient's vagus nerve. In one or more embodiments, the first electrical stimulation lead 1001 may extend from the IPG 800 to the left branch of the patient's hypoglossal nerve (e.g., a proximal or distal portion of the left branch of the hypoglossal nerve) and the second electrical stimulation lead 1002 may extend from the IPG 800 to the left side of the patient's vagus nerve. In one or more embodiments, the first electrical stimulation lead 1001 may extend from the IPG 800 to the left branch of the patient's hypoglossal nerve (e.g., a proximal or distal portion of the left branch of the hypoglossal nerve) and the second electrical stimulation lead 1002 may extend from the IPG 800 to the patient's left phrenic nerve. In one or more embodiments, the first electrical stimulation lead 1001 may extend from the IPG 800 to the right branch of the patient's hypoglossal nerve (e.g., a proximal or distal portion of the right branch of the hypoglossal nerve) and the second electrical stimulation lead 1002 may extend from the IPG 800 to the patient's right phrenic nerve. In one or more embodiments, the first and second electrical stimulation leads 1001, 1002 may be configured to provide electrical stimulation to one or more areas of the patient selected from the vagus nerve, the hypoglossal nerve, the spinal cord, nerves in the neck such as occipital nerves, and peripheral nerves in the arms and legs.

In one or more embodiments, the first and second electrical stimulation leads 1001, 1002 may be configured to provide bilateral stimulation to the patient. For instance, in one or more embodiments, the first electrical stimulation lead 1001 may extend from the IPG 800 to the left branch of the patient's hypoglossal nerve (e.g., a proximal or distal portion of the left branch of the hypoglossal nerve) and the second electrical stimulation lead 1002 may extend from the IPG 800 to the right branch of the patient's hypoglossal nerve (e.g., a proximal or distal portion of the right branch of the hypoglossal nerve). In one or more embodiments, the first electrical stimulation lead 1001 may extend from the IPG 800 to the left side of the patient's vagus nerve and the second electrical stimulation lead 1002 may extend from the IPG 800 to the right side of the patient's vagus nerve. In one or more embodiments, the first electrical stimulation lead 1001 may extend from the IPG 800 to the patient's left phrenic nerve and the second electrical stimulation lead 1002 may extend from the IPG 800 to the patient's right phrenic nerve.

The clinician can determine what configuration a particular IPG is being used with among different lead system configurations: (1) the IPG conventionally used with a stimulation lead having the same number of stimulation channels/electrode contacts or (2) the IPG is used with a splitter and two stimulation leads.

While the clinician can determine what the stimulation system configuration is being used by reviewing the patient records, or, in some cases, by palpating the presence of and the location of stimulation leads, or by scanning the body, e.g. with X-rays, it would be desirable for the IPG 800 to automatically detect among lead system configurations to at least determine between: (1) an IPG used with a single stimulation lead (as shown in FIG. 1) and (2) and an IPG 800 used with a lead splitter 900 and two stimulation leads 1001, 1002 (as shown in FIG. 3). The IPG 800 and/or clinician programmer device 1100 should automatically detect the system lead configuration and visually display the lead configuration on the clinician programmer screen 1101 or the patient remote screen 1201. This may be accomplished by using the embodiment of the lead splitter 900 described herein and using sensing circuitry and software programming in the IPG 800 and/or clinician programmer 1100 to detect the presence of the lead splitter 900.

In some embodiments of the lead splitter adapter 900, two or more electrical contacts in the first and second receptacles of the lead splitter are shorted (shunted) and the IPG 800 is configured to measure or determine the impedance between electrical contacts on the plug portion of the adapter. By determining impedance between pairs of electrical contacts at the plug end of the lead splitter adapter 900, the IPG 800 can automatically detect between: (1) a single lead connected to the IPG (shown in FIG. 1) or (2) a lead splitter adapter 900 connected to two stimulation leads 1001, 1002 (shown in FIG. 3). In this manner, the resistance, capacitance, and/or inductance of the lead contacts of the lead splitter adapter 900 encodes an identification (ID) used by the IPG 800 to indicate the configuration of the lead splitter adapter 900 and/or the lead(s) 1001, 1002.

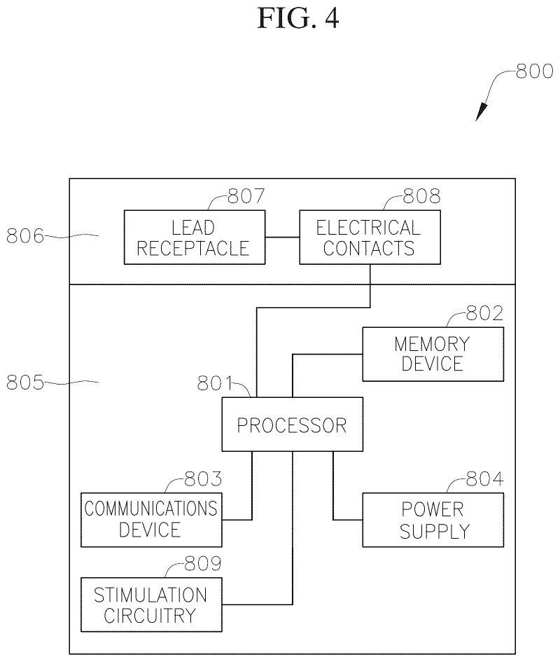

FIG. 4 shows a block diagram of an embodiment of the IPG 800. The IPG 800 includes a processor (e.g., a processing circuit) 801, a non-volatile memory device 802 (e.g., flash memory), a communications device 803 (e.g., a receiver and a transmitter, or a transceiver), and a power supply 804 (e.g., a primary battery or an inductively chargeable rechargeable battery). The communications device 803 provides wireless communication links through the skin of the patient to the CP device 1100 and the PR device 1200. Wireless links may include Bluetooth™, Bluetooth Low Energy or other protocols with suitable authentication and encryption to protect patient data. In one or more embodiments, the non-volatile memory device 802, the communications device 803, and the power supply 804 are in communication with each other over the processor 801. Additionally, in the illustrated embodiment, the processor 801, the non-volatile memory device 802, the communications device 803, and the power supply 804 are housed in a housing or a case 805. The case 805 includes a header 806 (e.g., a top epoxy part) and a lead receptacle 807 (e.g., a port or opening) in the header 806. The header 806 also includes a plurality of electrical contacts 808 (e.g., annular (ring-shaped) contacts) in the lead receptacle 807. The electrical contacts 808 may include any suitably conductive metal, such as titanium or a stainless-steel alloy. Typically, the electrical contacts inside the lead receptacle 807 in the IPG header 806 are canted coil springs that are shaped into rings which accept the plug or connector end of the lead. An example of such a canted coil spring connector is described in U.S. Pat. No. 10,535,945, the entire content of which is incorporated herein by reference. In one embodiment, the header 806 of the IPG 800 includes six electrical contacts 808 in the lead receptacle 807, although in one or more embodiments the header 806 of the IPG 800 may include any other suitable number of electrical contacts 808 in the lead receptacle 807. The electrical contacts 808 are connected to dedicated stimulation circuitry 809 in the IPG 800 that provides stimulation pulses as controlled by the processor 801.

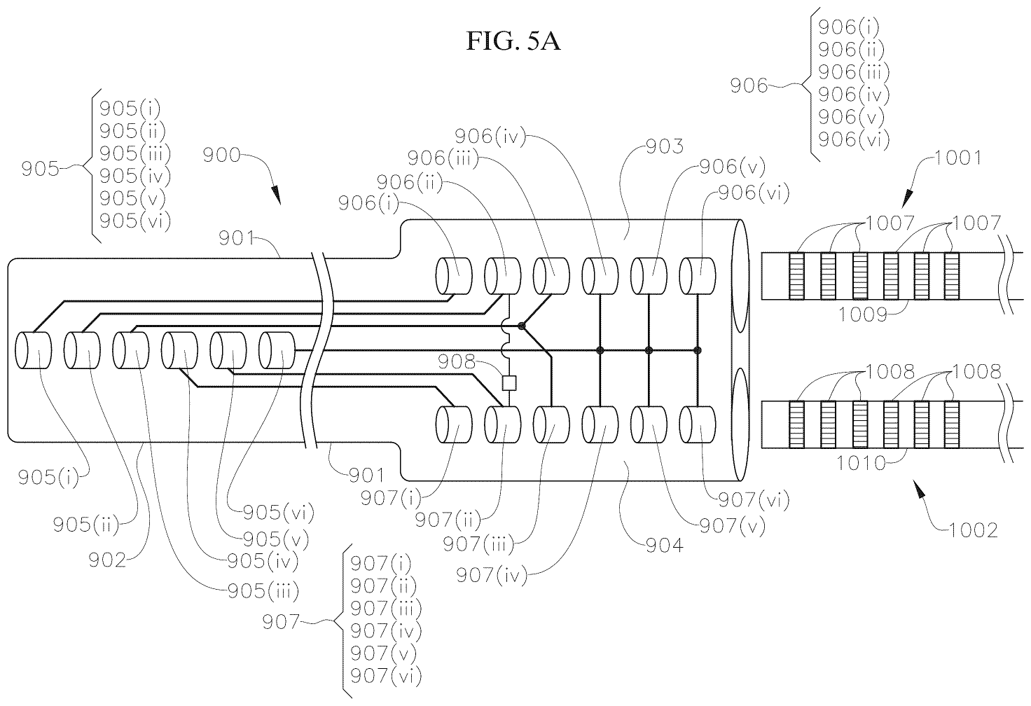

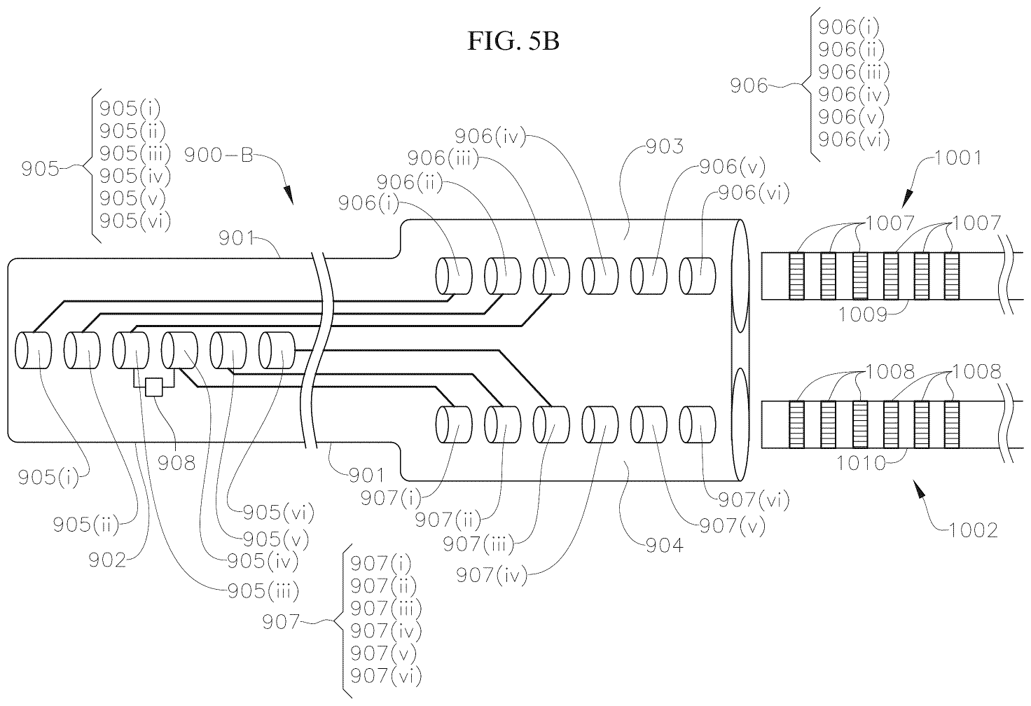

FIG. 5A depicts a lead splitter 900 according to one embodiment of the present disclosure. This lead splitter 900 allows a six-channel IPG 800 to be connected to two stimulation leads 1001, 1002 that each have six connector contacts at their proximal ends and six electrode contacts at their distal ends. The six-channel IPG 800 has six electrical contacts 808 located within the lead receptacle 807. The IPG 800 may be programmable so that each of the six electrical contacts 808 may function independently as either a negative contact or a positive contact, or be turned OFF. In some embodiments, the IPG 800 may use a metal portion of the housing 805 as a positive, anode (or return) electrode. The use of the lead splitter 900 enables the use of two, six-electrode contact stimulation leads in a bifurcated configuration. The lead splitter 900, in turn, is connected to the six-channel lead receptacle 807 of the IPG 800. The example IPG 800 shown in FIG. 4 has a single lead receptacle 807. It will be understood that the IPG 800 may have a different number of channels other than six channels. For example, the IPG may have four or eight channels within one lead receptacle 807. The IPG 800 may also have multiple lead receptacles, e.g., two, three, or four lead receptacles. The splitter 900 may be connected to one of the lead receptacles of an IPG having multi-lead receptacles.

The lead splitter 900 includes a body 901 having a plug portion 902 at a proximal end of the body 901 and first and second receptacle portions 903, 904 at a distal end of the body 901. The plug portion 902 of the lead adapter 900 is configured to plug into the lead receptacle 807 in the IPG 800, and the first and second receptacle portions 903, 904 of the lead splitter 900 are configured to receive the proximal ends 1009, 1010 of the first and second electrical stimulation leads 1001, 1002, respectively.

In the illustrated embodiment, the lead splitter 900 includes a plurality of electrical contacts 905 at the plug portion 902 (e.g., a plurality of electrical contacts 905 exposed on an outer surface of the plug portion 902). In one or more embodiments, the number of electrical contacts 905 at the plug portion 902 is equal to the number of electrical contacts 808 in the lead receptacle 807 of the IPG 800. Although in the illustrated embodiment the lead splitter 900 includes six electrical contacts 905(i)-905(vi) the lead splitter 900 may include any other suitable number of electrical contacts 905 depending on the number of electrical contacts 808 in the lead receptacle 807 of the IPG 800. When the plug portion 902 of the lead splitter 900 is plugged into the lead receptacle 807 of the IPG 800, the electrical contacts 905 of the lead splitter 900 contact the electrical contacts 808 in the lead receptacle 807 of the IPG 800.

In the illustrated embodiment, the lead splitter 900 also includes a plurality of electrical contacts 906 in the first receptacle portion 903 and a plurality of electrical contacts 907 in the second receptacle portion 904. Although in the illustrated embodiment the first and second receptacle portions 903, 904 of the lead splitter 900 each include six electrical contacts 906(i)-906(vi) and 907(i)-907(vi), respectively, the first and second receptacle portions 903, 904 of lead splitter 900 may include any other suitable number of electrical contacts 906, 907.

As shown in FIG. 5A, the lead splitter plug portion 902 connects to the IPG lead receptacle 807 which has six electrical contacts 808. Each of the two splitter lead receptacles 903, 904 accepts the proximal connector end 1009, 1010 of a stimulation lead 1001, 1002 that has six electrical contacts 1007, 1008, respectively. Normally, a single such stimulation lead would fit directly into the lead receptacle 807 of the six-stimulation channel IPG 800. This lead splitter 900 enables a bifurcated stimulation lead configuration so that stimulation can be delivered to two separate nerves in the body. The design of the lead splitter 900 also allows the IPG 800 to automatically detect whether the lead configuration is in a bifurcated stimulation lead mode or a single stimulation lead mode. In one or more embodiments, the lead splitter 900 includes an electrical connection (shunt) 908 between one of the electrical contacts (e.g., electrical contact 906(ii)) in the set of electrical contacts 906(i)-906(vi) of the first receptacle portion 903 and one of the electrical contacts (e.g., electrical contact 907(ii)) of the set of electrical contacts 907(i)-907(vi) of the second receptacle portion 904. For the embodiment shown in FIG. 5A, the alternative option is to shunt the electrical connection (shunt) 908 between electrical contact 906(i) and electrical contact 907(i). The electrical connection (shunt) 908 presents a detectable electrical signature (e.g., an impedance, a resistance, an inductance, or a capacitance), which can be detected by the IPG 800 and thereby indicate that the IPG 800 is connected to the lead splitter 900, instead of directly to a stimulation lead (e.g., the stimulation lead 300 or one of the stimulation leads 1001 or 1002). Although in the embodiment of FIG. 5A the electrical connection 908 is a shunt between electrical contacts 906(ii) and 907(ii), in one or more embodiments the electrical connection 908 may be a shunt between any other electrical contacts 906(i)-906(vi) and 907(i)-907(vi) in the first and second receptacle portions 903, 904. Additionally, in one or more embodiments, the electrical connection 908 may be in the plug portion 902 as a shunt between two of the electrical contacts 905(i)-905(vi) (e.g., between electrical contacts 905(ii) and 905(v)) to provide an equivalent circuit to the one depicted in FIG. 5A. For example, placing the electrical connection (shunt) 908 between electrical contacts 905(ii) and 905(v) is equivalent to placing the electrical connection (shunt) 908 between 906(ii) and 907(ii). The fourth, fifth, and sixth electrical contacts 906(iv)-906(vi) in the first receptacle portion 903 are shorted to the fourth, fifth, and sixth electrical contacts 907(iv)-907(vi) in the second receptacle portion 904 and also connected altogether to electrical contact 905(vi). Electrical contacts 906(iii), 907(iii), and 905(iii) are all connected together. The third, fourth, fifth, and sixth electrical contacts 906(iii)-906(vi) and 907(iii)-907(vi) in each of the first and second receptacle portions 903, 904 may be electrically connected to electrode contacts and may be selected as and typically function only as anodes in the first and second stimulation leads 1001, 1002. Accordingly, in one or more embodiments, only the first and second electrical contacts 906(i)-906(ii) and 907(i)-907(i) in each of the first and second receptacle portions 903, 904 are “active” electrical contacts, i.e., selectable as cathode, anode, or OFF and configured to deliver electrical stimulation to the electrode contacts 1003, 1004 (shown in FIG. 1) of the electrical stimulation leads 1001, 1002 in either unipolar or bipolar stimulation modes as previously described. Each of the corresponding electrode contacts 1003, 1004 on the stimulation leads 1001, 1002 connected to the electrical contacts 906(i)-906(ii) and 907(i)-907(ii) may function singly or concurrently as cathodes. The other electrode contacts that are not used will be programmed OFF or be inactive. Each stimulation electrode contact 1003, 1004 can independently deliver a stimulus pulse, for example, having a different stimulus pulse amplitude, delivered in either constant current or constant voltage. In one or more embodiments, any other of the electrical contacts 906 and 907 may be shorted (with a conductor) or shunted (with the electrical connection 908) depending on the number of electrical contacts 1007, 1008 at the proximal ends 1009, 1010 of the electrical stimulation leads 1001, 1002. In one or more embodiments, the number of shorted electrical contacts 906, 907 of the lead splitter 900 is equal to the difference between the number of electrical contacts 808 of the IPG 800 and the number of electrical contacts 1007, 1008 at the proximal end 1009, 1010 of each of the electrical stimulation leads 1001, 1002 (e.g., in an embodiment in which the IPG 800 includes six electrical contacts 808 and each of the electrical stimulation leads 1001, 1002 includes two electrical contacts 1007, 1008, four of the electrical contacts 906, 907 in each of the first and second receptacle portions 903, 904 of the lead splitter 900 are shorted; in an embodiment in which the IPG 800 includes six electrical contacts 808 and each of the electrical stimulation leads 1001, 1002 includes three active electrical contacts 1007, 1008, three of the electrical contacts 906, 907 in each of the first and second receptacle portions 903, 904 of the lead adapter 900 are shorted; and in an embodiment in which the IPG 800 includes five electrical contacts 808 and each of the electrical stimulation leads 1001, 1002 includes two active electrical contacts 1007, 1008, three of the electrical contacts 906, 907 in each of the first and second receptacle portions 903, 904 of the lead adapter 900 are shorted.

FIG. 5B depicts a lead splitter 900B according to another embodiment of the present disclosure. All of the components of the lead splitter 900 shown in FIG. 5A are present in the lead splitter 900B with the following exceptions: the subset of contacts 906(iv), 906(v), 906(vi), 907(iv), 907(v) and 907(vi) are, in this example, merely mechanical contacts and not electrical contacts (i.e., there is no electrical connection or shunt from any one of these contacts 906(iv), 906(v), 906(vi), 907(iv), 907(v) and 907(vi) to another contact within the subset of contacts 906(iv), 906(v), 906(vi), 907(iv), 907(v) and 907(vi)). Additionally, in this example, there is an electrical connection (shunt) 908 between electrical contacts 905(iii) and 905(iv) in the plug portion 902 of the lead splitter 900B. The electrical connection 908 can also be made between electrical contacts 906(iii) and 907(i), providing an equivalent electrical circuitry, but having the electrical connection 908 placed in the first and second receptacles 903, 904 in the lead splitter 900B rather than in the plug portion 902. The electrical connection 908 may include at least one of the following electrical components: a resistor, a capacitor, and/or an inductor and provide an overall impedance. Although this example shows an electrical connection 908 between electrical contacts 905(iii) and 905(iv), the placement of the electrical connection 908 may be chosen between any one contact in the subset of contacts 905(i)-(iii) and any one contact in the subset of contacts 905(iv)-(vi). As an equivalent from an electric circuit perspective, electrical connection 908 may be placed in the receptacle portion 903, 904 of the lead splitter 900B, connecting one contact selected from the subset of contacts 906(i)-(iii) to one contact selected from the subset of contacts 907(i)-(iii). The purpose of having the electrical connection 908 is to present (e.g., generate or produce) a signature impedance (resulting from a connection having at least one of a resistor, capacitor, or inductor) that indicates that the IPG 800 is connected to the lead splitter 900B, as opposed to being directed connected to a stimulation lead (e.g., stimulation lead 300). When the IPG 800 measures or detects impedance between electrical contacts 905(iii) and 905(iv) and connection 908, compared to, for example, the impedance measured between non-shunted electrical contacts 905(i) and 905(ii), the two impedances will be substantially different and therefore automatically indicate that the IPG 800 is connected to a lead splitter 900B, instead of directly to a stimulation lead (e.g., stimulation lead 300). The impedance between contacts 905(iii) and 905(iv), e.g. can be made to be less than approximately 100 ohms and this will be substantially less than the impedance measured between non-shunted electrical contacts 905(i) and 905(ii), which may be greater than approximately 1500 ohms when the lead splitter 900B and the two connected stimulation leads 1001, 1002 are implanted in the body. Implanted stimulation leads directly connected to an IPG can, for example, present impedances over approximately 1000 ohms, when measured between electrical contacts at the proximal end of the lead.

Referring again to FIG. 4, showing the block diagram of the IPG 800, in one or more embodiments, the non-volatile memory device 802 of the IPG 800 includes computer-readable instructions (e.g., software code) that, when executed by the processor 801, cause the IPG 800 to determine which electrical contacts 906, 907 of the lead splitter adapter 900 are shorted. For instance, in one or more embodiments, the instructions stored in the memory device 802, when executed by the processor 801, cause the IPG 800 to deliver current from the power supply 804 to the lead splitter adapter 900 and to determine (e.g., measure or acquire) the impedance across the pairs of electrical contacts 905. Additionally, in one or more embodiments, the instructions, when executed by the processor 801, cause the IPG 800 to compare the measured impedance values to a threshold impedance value and to determine that those pairs of electrical contacts 905 that have an impedance value below the threshold impedance value are shorted (shunted). Additionally, in one or more embodiments, the non-volatile memory device 802 includes a lookup table associating different shorted electrical contacts 906, 907 with different configurations of the electrical stimulation leads 1001, 1002. For instance, in one or more embodiments, the shorted electrical contacts 906, 907 may indicate that the IPG 800 is connected to two electrical stimulation leads (i.e., bifurcated leads) and the number of electrode contacts on each of the leads. Accordingly, the manner in which the electrical contacts 906, 907 are shorted encodes identification information regarding the configuration of the electrical stimulation leads 1001, 1002.

Additionally, in one or more embodiments, the instructions stored in the memory device 802, when executed by the processor 801, cause the dedicated stimulation circuitry 809 of the IPG 800 to deliver stimulation to the electrodes 1003, 1004 of the electrical stimulation leads 1001, 1002 based on the configuration of the electrical stimulation leads that was determined according to the manner in which the electrical contacts 906, 907 of the lead splitter adapter 900 were shorted. For example, in one or more embodiments, the instructions stored in the memory device 802, when executed by the processor 801, cause the IPG 800 to change the mode of stimulation based on the configuration of the electrical stimulation leads 1001, 1002 that was determined according to the manner in which the electrical contacts 906, 907 of the lead splitter adapter 900 are shorted. Furthermore, in one or more embodiments, the instructions stored in the memory device 802, when executed by the processor, cause the communications device 803 of the IPG 200 to wirelessly transmit a signal to the CP device 1100 and/or to the PR device 1200 that causes a graphical user interface (GUI) displayed on the display 1101, 1201 of the CP device 1100 and/or to the PR device 1200 to display various information (e.g., the mode of operation of the IPG 800 and/or the configuration of the electrical stimulation leads 1001, 1002) based on the manner in which the electrical contacts 906, 907 of the lead adapter 900 are shorted.

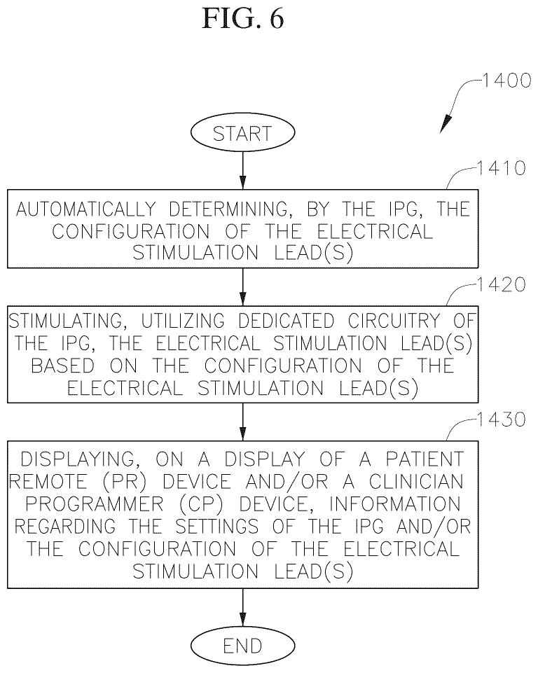

FIG. 6 shows a flowchart of a method for detecting a system lead configuration between (1) an IPG with a single stimulation lead and (2) an IPG with a lead splitter and two stimulation leads. The method 1400 includes a task 1410 of determining (e.g., automatically determining), by the IPG, the configuration of the electrical stimulation lead(s) that is attached to the IPG. In one or more embodiments, the task 1410 includes delivering current from the IPG electrical contacts in the lead receptacle and determining (e.g., measuring or calculating) the impedance or resistance across the electrical contacts on either the connected stimulation lead or the plug portion of the lead splitter 900 shown in FIG. 5, and comparing the measured impedance or resistance values to a threshold impedance value or a threshold resistance value, and determining that those two electrode contacts that have an impedance value (or a resistance value) below the threshold impedance value (or the threshold resistance value) are shunted via, e.g., the electrical connection and therefore automatically indicate connection of the IPG to the splitter. Alternatively, the task 1410 includes delivering current from the IPG electrical contacts in the lead receptacle and determining (e.g., measuring or calculating) the impedance across the electrical contacts 905(iii) and 905(iv) on the plug portion of the lead splitter 900B shown in FIG. 5B, and comparing the measured impedance values to a threshold impedance value, and determining if the impedance values obtained indicate that the IPG is connected to the lead adapter 900B instead of directly to a stimulation lead 300. In one or more embodiments, the task 1410 may also include referencing a lookup table (e.g., stored in the non-volatile memory device of the IPG), which associates different shunted electrodes with different configurations of the electrical stimulation lead, to determine the configuration of the electrical stimulation lead. In one or more embodiments, the task 1410 includes delivering a constant (or substantially constant) current from the IPG to the connected lead adapter 900, 900B or connected stimulation lead 300 as shown in FIG. 1 and determining (e.g., measuring or acquiring) the voltage between paired electrical contacts on the plug portion of the lead adapter or paired electrical contacts on the connector portion of the stimulation lead and thereby determine (e.g., measure or acquire) the impedance or resistance and location between two of the electrical contacts of the lead adapter or stimulation lead. In one or more embodiments, the task 1410 also includes referencing a lookup table (e.g., stored in the non-volatile memory device of the IPG), which associates different impedance or resistance values and/or different locations of the resistor with different configurations of the electrical stimulation lead, to determine the configuration of the electrical stimulation lead. In one or more embodiments, the task 1410 may determine that the IPG is connected to a single electrical stimulation lead or a pair of electrical stimulation leads (e.g., a pair of electrical stimulation leads configured to provide bifurcated stimulation to the patient).

In the illustrated embodiment, the method 1400 also includes a task 1420 of stimulating, utilizing dedicated circuitry of the IPG, the electrical stimulation lead(s) based on the configuration of the electrical stimulation lead(s) determined in task 1410 (e.g., setting a stimulation mode, e.g. bipolar or monopolar stimulation of the IPG based on the configuration of the electrical stimulation lead determined in task 1410).

In one or more embodiments, the method 1400 may include a task 1430 of displaying, on a display of a patient remote (PR) device and/or a clinician programmer (CP) device in wireless communication with the new IPG, information regarding the operating mode or settings of the IPG and/or the configuration of the electrical stimulation lead(s) determined in task 1410. The task 1430 may include transmitting a wireless signal, from the new IPG to the PR device and/or the CP device, which cause a graphical user interface (GUI) displayed on the display of the PR device and/or the CP device to display information regarding the mode or settings of the new IPG and/or the configuration of the electrical stimulation lead(s). In the manner described above, the method 1400 enables the new IPG to function with the existing electrical stimulation lead(s) implanted in the patient, even when the electrical stimulation lead(s) is/are physically incompatible with the new IPG. In one or more embodiments, in response to the task 1410 determining that the stimulation lead is directly connected to the IPG (i.e., the lead adapter is not utilized), the method 1430 may include displaying, on a display of a patient remote device or a clinician programmer device in wireless communication with the IPG, information regarding the stimulation lead directly connected to the IPG.

The implantable pulse generator and/or any other relevant devices or components according to embodiments of the present invention described herein may be implemented utilizing any suitable hardware, firmware (e.g. an application-specific integrated circuit), software, or a combination of software, firmware, and hardware. For example, the various components of the device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the exemplary embodiments of the present invention.

When a first element is described as being “coupled” or “connected” to a second element, the first element may be directly “coupled” or “connected” to the second element, or one or more other intervening elements may be located between the first element and the second element. As used herein, the singular forms “a”, “an” and “the” are intended to include the plural forms as well, unless the context clearly indicates otherwise. Further, the use of “may” when describing embodiments of the present disclosure refers to “one or more embodiments of the present disclosure.”

Although some embodiments of the present disclosure are disclosed herein, the present disclosure is not limited thereto, and the scope of the present disclosure is defined by the appended claims and equivalents thereof.

What is claimed is:

1. A lead splitter adapter for an implantable pulse generator, the lead splitter adapter comprising:
a plug portion configured to plug into a port of the implantable pulse generator, the plug portion comprising a first plurality of electrical contacts;
a first receptacle portion and a second receptacle portion connected to the plug portion, each of the first receptacle portion and the second receptacle portion comprising a second plurality of electrical contacts; and
an electrical connection that includes at least one of a resistor, an inductor, or a capacitor between two of the first plurality of electrical contacts of the plug portion or between one of the second plurality of electrical contacts of the first receptacle portion and one of the second plurality of electrical contacts of the second receptacle portion.

2. The lead splitter adapter of claim 1, wherein the electrical connection is a resistor that has a fixed resistance value.

3. A stimulator system comprising:
an implantable pulse generator comprising:
a header;
a lead receptacle in the header;
a plurality of electrical contacts in the lead receptacle;
a processor;
a non-volatile memory device; and
a power supply;
a lead splitter adapter configured to connect at least one electrical stimulation lead to the implantable pulse generator, the lead splitter adapter comprising:
a plug portion configured to extend into the lead receptacle in the header of the implantable pulse generator, the plug portion comprising a first plurality of electrical contacts; and
at least one receptacle portion connected to the plug portion, the at least one receptacle portion comprising a second plurality of electrical contacts, wherein the at least one receptacle portion is configured to receive a proximal end portion of the electrical stimulation lead,
wherein the non-volatile memory device comprises instructions which, when executed by the processor, cause the implantable pulse generator to determine at least one of a impedance, a resistance, a capacitance, or an inductance between two of the first plurality of electrical contacts of the plug portion and to determine a configuration of the at least one electrical stimulation lead based on the impedance, the resistance, the capacitance, or the inductance.

4. The stimulator system of claim 3, wherein at least one pair of electrical contacts of the second plurality of electrical contacts of the lead splitter adapter is shorted.

5. The stimulator system of claim 4, wherein the at least one pair of electrical contacts is electrically connected with a resistor between the at least one pair of electrical contacts.

6. The stimulator system of claim 3, wherein the instructions stored in the non-volatile memory device, when executed by the processor, cause the implantable pulse generator to deliver electrical stimulation based on the configuration of the at least one electrical stimulation lead.

7. The stimulator system of claim 3, wherein:
the at least one receptacle portion comprises a single receptacle portion, and
the instructions stored in the non-volatile memory device, when executed by the processor, cause the implantable pulse generator to determine that the configuration of the at least one electrical stimulation lead is a single electrical stimulation lead.

8. The stimulator system of claim 3, wherein:
the lead splitter adapter comprises a first receptacle portion and a second receptacle portion, and
the instructions stored in the non-volatile memory device, when executed by the processor, cause the implantable pulse generator to determine that the configuration of the at least one electrical stimulation lead is a pair of electrical stimulation leads configured to provide bilateral stimulation.

9. The stimulator system of claim 3, further comprising a patient remote device in electronic communication with the implantable pulse generator, and wherein the instructions stored in the non-volatile memory device, when executed by the processor, cause the implantable pulse generator to transmit a signal to the patient remote device, the signal being configured to cause the patient remote device to display information based on the configuration of the at least one electrical stimulation lead.

10. The stimulator system of claim 3, further comprising a clinician programmer device in electronic communication with the implantable pulse generator, and wherein the instructions stored in the non-volatile memory device, when executed by the processor, cause the implantable pulse generator to transmit a signal to the clinician programmer device, the signal being configured to cause the clinician programmer device to display information based on the configuration of the at least one electrical stimulation lead.

11. A method of identifying an IPG lead configuration, the method comprising:
providing a lead splitter comprising a proximal plug portion, a first lead receptacle, and a second lead receptacle;
connecting an IPG to the proximal plug portion of the lead splitter;
connecting a first stimulation lead to the first lead receptacle and a second stimulation lead to the second lead receptacle;
implanting the lead splitter, the first stimulation lead, the second stimulation lead, and the IPG into a body;
delivering current, from the IPG, to the lead splitter; and
determining or detecting an electrical signature of the lead splitter, the electrical signature comprising at least one of an impedance, a resistance, a conductance, or an inductance, the electrical signature indicating the lead splitter having an electrical connection shunt between a pair of electrical contacts of the proximal plug portion or between one electrical contact of the first lead receptacle and another electrical contact of the second lead receptacle.

12. A method for automatically configuring an IPG for a lead configuration, the method comprising:
determining whether the IPG is connected to a lead splitter or directly connected to a stimulation lead; and
in response to the lead splitter being detected, configuring an operating mode and/or settings of the IPG to accommodate the lead splitter and two stimulation leads, wherein the lead splitter comprises an electrical connection shunt that provides an electrical signature comprising at least one of one of an impedance, a resistance, a conductance, or an inductance that can be detected by the IPG, the electrical signature indicating that the lead splitter is connected to the IPG.

13. The method of claim 12, further comprising:
displaying, on a display of a patient remote device or a clinician programmer device in wireless communication with the IPG, information regarding the operating mode of the IPG, the settings of the IPG, and/or a configuration of the two stimulation leads.

14. The method of claim 12, further comprising:
displaying, on a display of a patient remote device or a clinician programmer device in wireless communication with the IPG, information regarding the stimulation lead directly connected to the IPG.
